(19) United States
(12) Patent Application Publication
(10) Pub. No.: US 2024/0105307 A1
(43) Pub. Date: Mar. 28, 2024

(54) METHOD FOR DETERMINING PERSONALIZED DHA SUPPLEMENTATION

(71) Applicant: SOCIETE DES PRODUITS NESTLE S.A., Vevey (CH)

(72) Inventors: CARLOS ANTONIO DE CASTRO, Singapore (SG); FANG HUANG, Beijing (CN); IRMA SILVA ZOLEZZI, Vevey (CH); SAGAR THAKKAR, Tresalveo (SG); JACKLYN RUILIN CHEN, Singapore (SG); SURABHI DEVARAJ, Fort Gardens (SG)

(21) Appl. No.: 18/263,550

(22) PCT Filed: Jan. 27, 2022
(86) PCT No.: PCT/EP2022/051921

§ 371 Date: Jul. 31, 2023

(30) Foreign Application Priority Data
Feb. 1, 2021 (EP) 21154485.3

(51) Int. Cl. G16H 20/60; G01N 33/92; G16H 20/10

(52) U.S. Cl. CPC G16H 20/60; G01N 33/92; G16H 20/10

(57) ABSTRACT

The present invention relates to a method for determining a personalised docosallexaenoic acid (DHA) supplementation dose to be taken by a mother to meet the DHA requirements of the mother's infant.

### FIELD OF THE INVENTION

The present invention relates to a method for determining a personalised docosahexaenoic acid (DHA) supplementation dose to be taken by a mother to meet the DHA requirements of the mother's infant.

### BACKGROUND TO THE INVENTION

Docosahexaenoic acid (DHA) is present in breast milk and plays an important role in early infant development. Long chain polyunsaturated fatty acids (LC-PUFAs), including DHA, are incorporated into membrane phospholipids and play a structural role in these membranes. LC-PUFAs also act as precursors of signalling molecules and as activators of a number of gene transcription factors. The essentiality of n-3 LC-PUFA is generally contributed to the incorporation of DHA in uniquely high levels in the central nervous system, although DHA is also incorporated in most other tissues where it may also have important functional effects (Lauritzen, L., et al., 2016. Nutrients, 8(1), p. 6).

Breast milk is the most important source of LC-PUFA during the first two years of life. However, the breast milk DHA content is very dependent on the mother's diet and can be very low in populations living on a primarily plant-based diet with no or limited fish intake (Agostoni, C., et al., 2016. Sight and Life, 30, pp. 82-6). Breast milk DHA content varies from 0.13 to 0.37 g DHA per 100 g fatty acids (Netherlands) up to up to a median level of 1.4 g DHA per 100 fatty acids (Inuit, Canada) (Stoutjesdijk, E., et al., 2018. Prostaglandins, Leukotrienes and Essential Fatty Acids, 128, pp. 53-61).

Maternal supplementation can be used to increase breast milk levels of DHA. DHA supplementation of mothers during the complementary feeding period may be effective in improving neurofunctional and visual performance (Agostoni, C., 2010. Early human development, 86(1), pp. 3-6). Moreover, a high milk DHA supply to very low birth weight infants may enhance early visual and cognitive development and reduce adverse events including severe developmental delay, bronchopulmonary dysplasia, necrotizing enterocolitis and allergies (Koletzko, B., 2017. Clinics in perinatology, 44(1), pp. 85-93).

However, despite the wide variation in breast milk DHA content, DHA supplementation recommendations are not typically personalised to the mother. Breastfeeding mothers are usually advised to achieve a minimum average daily intake of 200 mg DHA, which may be insufficient for some breastfeeding mothers to achieve a breast milk DHA content which can meet the requirements of the mother's infant.

Thus, there is a demand for new personalised DHA supplementation doses.

### SUMMARY OF THE INVENTION

The inventors have developed a new method for determining a personalised DHA supplementation dose to be taken by a breastfeeding mother.

The inventors have surprisingly found that maternal parameters (such as the mother's milk DHA level and the mother's BMI) and infantile parameters (such as infant weight-for-age z-score and infant age) can be used to determine the mother's personalised DHA supplementation dose. The dependency between dietary intake amount of DHA and such parameters can be determined for a population using regression analysis and used to reliably determine a personalised DHA supplementation dose for a mother from that population.

In one aspect, the present invention provides a method for determining a personalised DHA supplementation dose to be taken by a mother to meet the DHA requirements of the mother's infant, wherein the method comprises using one or more parameters to determine the personalised DHA supplementation dose, preferably wherein the one or more parameters comprise the mother's milk DHA level and one or more infantile parameters.

In some embodiments, the one or more infantile parameters comprise infant weight-for-age z-score and/or infant age. In some embodiments, the method further comprises using maternal BMI to determine the personalised DHA supplementation dose. In some embodiments, the method comprises using mother's milk DHA level, maternal BMI, infant weight-for-age z-score and infant age to determine the personalised DHA supplementation dose.

In some embodiments, the method further comprises using one or more additional parameters to determine the personalised DHA supplementation dose, preferably wherein the one or more additional parameters are selected from one or more of the country or region, genetic data, maternal age, dietary data, and lifestyle data.

In some embodiments, the DHA requirements of the mother's infant is a mother's milk DHA level of at least 0.3 g DHA per 100 g fatty acids, at least 0.37 g DHA per 100 g fatty acids, or at least 0.7 g DHA per 100 g fatty acids. In some embodiments, the DHA requirements of the mother's infant is a mother's milk DHA level of 0.3-1.0 g DHA per 100 g fatty acids, 0.37-1.0 g DHA per 100 g fatty acids, 0.7-1.0 g DHA per 100 g fatty acids, or about 0.7 g DHA per 100 g fatty acids, preferably wherein the DHA requirement of the mother's infant is a mother's milk DHA level of about 0.7 g DHA per 100 g fatty acids.

The method for determining a personalised DHA supplementation dose to be taken by a mother to meet the DHA requirements of the mother's infant may comprise:
(i) using one or more parameters, preferably the mother's milk DHA level and one or more infantile parameters, to determine the mother's current DHA intake and/or the mother's target DHA intake; and
(ii) using the mother's current DHA intake and the mother's target DHA intake to determine the personalised DHA supplementation dose.

Suitably, the method comprises using mother's milk DHA level, maternal BMI, infant weight-for-age z-score and infant age, and optionally maternal BMI to determine the mother's current DHA intake and/or the mother's target DHA intake. In some embodiments, the method further comprises using one or more additional parameters to determine the mother's current DHA intake and/or the mother's target DHA intake, preferably wherein the one or more additional parameters are selected from one or more of the country or region, genetic data, maternal age, dietary data, and lifestyle data.

Suitably, the mother's current DHA intake and/or the mother's target DHA intake are determined using a regression equation, preferably wherein the regression equation is a multiple linear regression equation. Suitably, the regression equation is:

log(mother's current DHA intake or mother's target DHA intake)=a+b1*log(mother's milk DHA level or mother's target milk DHA level)+b2*log(maternal BMI)+b3*infant weight-for-age z-score+b4*infant age

wherein a, b1, b2, b3, b4 are determined by multiple linear regression.

For example, the mother's current DHA intake may be determined using:

log(mother's current DHA intake)=a+b1*log(mother's milk DHA level)+b2*log(maternal BMI)+b3*infant weight-for-age z-score+b4*infant age

For example, the mother's target DHA intake may be determined using:

log(mother's target DHA intake)=a+b1*log(mother's target milk DHA level)+b2*log(maternal BMI)+b3*infant weight-for-age z-score+b4*infant age

Suitably, the personalised DHA supplementation dose is determined using the difference between the mother's target DHA intake and the mother's current DHA intake. For example, the personalised DHA supplementation dose may be determined using the following equation:

personalised DHA supplementation dose=(mother's target DHA intake−mother's current DHA intake)*conversion rate.

In some embodiments, the conversion rate is from about 2 to about 3, or about 2.5. Preferably, the conversion rate is about 2.5.

In some embodiments, the personalised DHA supplementation dose is a daily dose. In some embodiments, the personalised DHA supplementation dose is rounded up to the nearest 5 mg, 10 mg, 20 mg, 25 mg, 50 mg, 100 mg, or 200 mg, preferably wherein the personalised DHA supplementation dose is rounded up to the nearest 200 mg.

In another aspect, the present invention provides a method for determining a personalised DHA supplementation dose to be taken by a mother to meet the DHA requirements of the mother's infant, wherein the method comprises:
(i) determining the mother's milk DHA level; and
(ii) determining the personalised DHA supplementation dose using the method of the invention.

In another aspect, the present invention provides a method for providing a personalised DHA supplementation dose to be taken by a mother to meet the DHA requirements of the mother's infant, wherein the method comprises:
(i) determining the personalised DHA supplementation dose using the method of the invention; and
(ii) administering the personalised DHA supplementation dose to the mother.

In another aspect, the present invention provides a method of improving the health and/or development of an infant, the method comprising administering a personalised DHA supplementation dose to the infant's mother, wherein the personalised DHA supplementation dose is determined using the method of the invention.

In another aspect, the present invention provides a personalised DHA supplementation dose, for use in a method of promoting development and/or improving the health of an infant, the method comprising administering a personalised DHA supplementation dose to the infant's mother, wherein the personalised DHA supplementation dose is determined using the method of the invention.

In some embodiments, the cognitive development, visual development, motor skill development and/or immune system development of the infant is improved. In some embodiments, the infant's risk of becoming overweight or obese in infancy and/or early childhood is reduced.

In another aspect, the present invention provides a computer program comprising instructions which, when the program is executed by a computer, cause the computer to carry out the method of the invention.

In another aspect, the present invention provides a computer program comprising instructions which, when the program is executed by a computer, cause the computer to determine a personalised DHA supplementation dose to be taken by a mother to meet the DHA requirements of the mother's infant, given one or more parameters, preferably wherein the one or more parameters are the mother's milk DHA level and one or more infantile parameters.

In another aspect, the present invention provides a computer-readable medium comprising instructions which, when executed by a computer, cause the computer to carry out the method of the invention.

In another aspect, the present invention provides a computer-readable medium comprising instructions which, when executed by a computer, cause the computer to determine a personalised DHA supplementation dose to be taken by a mother to meet the DHA requirements of the mother's infant, given one or more parameters, preferably wherein the one or more parameters are the mother's milk DHA level and one or more infantile parameters.

In another aspect, the present invention provides for use of a personalised DHA supplement, wherein the personalised DHA supplement dose is determined using the method of the invention.

### DESCRIPTION OF DRAWINGS

FIG. 1—weight for age z-score

Reproduced from WHO Child Growth Standards. Z-score expresses the anthropometric value as a number of standard deviations below or above the reference value, it is calculated from baby's weight, sex and accordingly standard deviation from WHO Child Growth Standards. (A) weight for age z-score for girls. (B) weight for age z-score for boys.

FIG. 2—Example of algorithm application

Baseline human milk DHA; infant sex, weight and age; and maternal BMI are used to determine a personalised DHA supplementation dose.

FIG. 3—model validation

Data collected from clinical trial done in other countries (China, Switzerland) instead of the seven countries mentioned in the first section, is fed into the built model for DHA supplementation dose prediction. (A) China. (B) Switzerland. (C) France, Italy, Norway, Portugal, Romania, Spain, and Sweden.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

### Method for Determining a Personalised DHA Supplementation Dose

In one aspect, the present invention provides a method for determining a personalised DHA supplementation dose to be taken by a mother.

### DHA Requirements of the Mother's Infant

A personalised DHA supplementation dose can be used to increase breast milk levels of DHA to meet the DHA requirements of the mother's infant.

The accumulation of DHA in the brain takes place during the brain growth spurt in the intrauterine and neonatal period up to two years of age. Due to the lack of de novo LC-PUFA synthesis, the rate of membrane DHA incorporation in early life, in the brain as well as in other tissues, depends on direct maternal transfer, and then dietary supply (i.e., breastfeeding) and endogenous LC-PUFA production. Breast milk is the most important source of LC-PUFA during the first two years of life (Agostoni, C., et al., 2016. Sight and Life, 30, pp. 82-6).

The method of the present invention can be used to determine a personalised DHA supplementation dose to be taken by a mother to meet any DHA requirement. For example, whilst human milk globally has an average DHA content of about 0.3% of fatty acid, to match intrauterine DHA accretion, very low birth weight infants (VLBWI) require a human milk DHA content of about 1% of fatty acid (Koletzko, B., 2017. Clinics in perinatology, 44(1), pp. 85-93).

The present invention can be used to meet a DHA requirement of about 0.3 g DHA per 100 g fatty acids, about 0.4 g DHA per 100 g fatty acids, about 0.5 g DHA per 100 g fatty acids, about 0.6 g DHA per 100 g fatty acids, about 0.7 g DHA per 100 g fatty acids, about 0.8 g DHA per 100 g fatty acids, about 0.9 g DHA per 100 g fatty acids, or about 1.0 g DHA per 100 g fatty acids.

In some embodiments, the DHA requirements of the mother's infant is a mother's milk DHA level of at least 0.3 g DHA per 100 g fatty acids, at least 0.37 g DHA per 100 g fatty acids, or at least 0.7 g DHA per 100 g fatty acids.

In some embodiments, the DHA requirements of the mother's infant is a mother's milk DHA level of 0.3-1.0 g DHA per 100 g fatty acids, 0.37-1.0 g DHA per 100 g fatty acids, 0.7-1.0 g DHA per 100 g fatty acids, or about 0.7 g DHA per 100 g fatty acids.

In some embodiments, the DHA requirement of the mother's infant is a mother's milk DHA level of about 0.7 g DHA per 100 g fatty acids.

### Maternal and Infantile Parameters

The method of the present invention comprises using one or more parameters to determine the personalised DHA supplementation dose. Suitably, the one or more parameters are used to determine the mother's current DHA intake and/or the mother's target DHA intake which are subsequently used to determine the personalised DHA supplementation dose.

In some embodiments, the method of the present invention comprises using two or more parameters (e.g. 2, 3, or 4 parameters) to determine the personalised DHA supplementation dose. In some embodiments, the method of the present invention comprises using three or more parameters to determine the personalised DHA supplementation dose. In some embodiments, the method of the present invention comprises using four or more parameters to determine the personalised DHA supplementation dose.

Exemplary parameters include mother's milk DHA level, mother's target milk DHA level, infant weight-for-age z-score, infant age, maternal BMI, country or region, genetic data, maternal age, dietary data, lifestyle data and the like.

In some embodiments, the method of the present invention comprises using one or more maternal parameter and one on more infantile parameter to determine the personalised DHA supplementation dose. For example, the method of the present invention may comprise using one or more maternal parameter and one on more infantile parameter to determine the mother's current DHA intake and/or the mother's target DHA intake.

As used herein, a “maternal parameter” includes any parameter or variable derivable from maternal data, such as demography, anthropometry, medical history, history of dietary supplements, and food diaries. A “maternal parameter” also includes any parameter or variable derivable from human milk samples. Exemplary maternal parameters include mother's milk DHA level and maternal BMI.

As used herein, an “infantile parameter” includes any parameter or variable derived from infant data such as demography, anthropometry, history of medication use, body composition, and infant HM intake diary. Exemplary infantile parameters include infant weight-for-age z-score and infant age.

In some embodiments, the method of the present invention comprises or consists of using the mother's milk DHA level and one or more infantile parameters to determine the personalised DHA supplementation dose. For example, in some embodiments the method of the present invention may comprise or consist of using the mother's milk DHA level and one or more infantile parameters to determine the mother's current DHA intake and/or the mother's target DHA intake.

In some embodiments, the method of the present invention comprises or consists of using the mother's milk DHA level and infant weight-for-age z-score and/or infant age to determine the personalised DHA supplementation dose. For example, in some embodiments the method of the present invention may comprise or consist of using the mother's milk DHA level and infant weight-for-age z-score and/or infant age to determine the mother's current DHA intake and/or the mother's target DHA intake.

Optionally, the method further comprises or further consists of using the maternal BMI to determine the personalised DHA supplementation dose. For example, optionally, the method further comprises or further consists of using the maternal BMI to determine the mother's current DHA intake and/or the mother's target DHA intake.

In some embodiments, the method of the present invention comprises or consists of using the mother's milk DHA level, maternal BMI, infant weight-for-age z-score and infant age to determine the personalised DHA supplementation dose. For example, in some embodiments the method of the present invention may comprise or consist of using the mother's milk DHA level, maternal BMI, infant weight-for-age z-score and infant age to determine the mother's current DHA intake and/or the mother's target DHA intake.

### Mother's Milk DHA Level

The method of the present invention preferably comprises using the mother's milk DHA level (i.e. the mother's current milk DHA level) to determine the personalised DHA supplementation dose. Suitably, the mother's milk DHA level is used to determine the mother's current DHA intake.

The method of the present invention may comprise determining the mother's milk DHA level prior to determining the personalised DHA supplementation dose. The method of the present invention may comprise determining the mother's milk DHA level and using this to determine the personalised DHA supplementation dose.

The mother's milk DHA level may be determined by any method known in the art. For example, a milk sample may be collected from the mother and the fatty acids analysed. Suitable methods for determining the mother's milk DHA level are described in Jackson, K. H., et al., 2016. International breastfeeding journal, 11(1), pp. 1-7 and Rudolph, M. C., et al., 2016. Journal of mammary gland biology and neoplasia, 21(3-4), pp. 131-138.

The milk sample may be stored prior to determining the DHA level. For example, the milk may be frozen, then thawed and homogenised prior to determining the DHA level or a drop of milk may be placed on filter paper and dried.

The fatty acids may be extracted prior to analysing the fatty acid. Any method for extracting fatty acids may be used. For example, the method described in Folch, et al. Journal of biological chemistry 226, no. 1 (1957): 497-509).

Gas chromatography or gas-liquid chromatography may be used to analyse the fatty acids. For example, the method described in AOCS Official Method Ce 1 h-05 may be used.

The mother's milk DHA level may be determined at any time during which the mother is breastfeeding the infant. Suitably, the mother's milk DHA level may be determined at any time between 0-2 years post-partum, or about 0-120 days post-partum. In some embodiments, the mother's milk DHA level is the mother's milk DHA level in the mother's first milk sample collected post-partum. For example, the mother's milk DHA level may be determined at about 0-3 days post-partum (e.g. about 0 days post-partum, about 1 day post-partum, about 2 days post-partum, or about 3 days post-partum). Suitably, the mother's milk DHA level may be determined at about 2 days post-partum.

The method of the present invention preferably comprises using the mother's target milk DHA level to determine the personalised DHA supplementation dose. Suitably, the mother's target DHA intake is determined using a mother's target milk DHA level. Subsequently this may be used with the mother's current DHA intake to determine the personalised DHA supplementation dose.

The “mother's target milk DHA level” is the mother's milk DHA level required to meet or exceed the DHA requirements of the mother's infant.

The method of the present invention can be used for any mother's target milk DHA level. In some embodiments, the mother's target milk DHA level exceeds the DHA requirements of the mother's infant. This may maximise the chance for the mother's milk DHA level to reach and/or exceed the DHA requirements of the mother's infant.

The mother's target milk DHA level may be a mother's milk DHA level of about 0.3 g DHA per 100 g fatty acids, about 0.4 g DHA per 100 g fatty acids, about 0.5 g DHA per 100 g fatty acids, about 0.6 g DHA per 100 g fatty acids, about 0.7 g DHA per 100 g fatty acids, about 0.8 g DHA per 100 g fatty acids, about 0.9 g DHA per 100 g fatty acids, or about 1.0 g DHA per 100 g fatty acids.

In some embodiments, the mother's target milk DHA level may be a mother's milk DHA level of at least 0.3 g DHA per 100 g fatty acids, at least 0.37 g DHA per 100 g fatty acids, or at least 0.7 g DHA per 100 g fatty acids.

In some embodiments, the mother's target milk DHA level may be a mother's milk DHA level of 0.3-1.0 g DHA per 100 g fatty acids, 0.37-1.0 g DHA per 100 g fatty acids, 0.7-1.0 g DHA per 100 g fatty acids, or about 0.7 g DHA per 100 g fatty acids.

In some embodiments, the mother's target milk DHA level may be a mother's milk DHA level of about 0.7 g DHA per 100 g fatty acids.

The mother's milk DHA level may be given in any measurement unit, suitably g DHA per 100 g fatty acids.

### Infant Weight-for-Age Z-Score

The method of the present invention may comprise using the infant weight-for-age z-score to determine the personalised DHA supplementation dose. Suitably, the infant weight-for-age z-score is used to determine the mother's current DHA intake and/or the mother's target DHA intake, which are subsequently used to determine the personalised DHA supplementation dose

The method of the present invention may comprise determining the infant weight-for-age z-score prior to determining the personalised DHA supplementation dose. The method of the present invention may comprise determining the infant weight-for-age z-score and using this to determine the personalised DHA supplementation dose.

The infant weight-for-age z-score may be determined by any method known in the art. For example using the weight-for-age toolkits provided by the World Health Organization (WHO) as described in WHO Multicentre Growth Reference Study Group, 2006. WHO Child Growth Standards based on length/height, weight and age. Acta paediatrica (Oslo, Norway: 1992). Supplement, 450, p. 76. Suitably, a LMS method can be used to determine the weight-for-age z-score as described in the WHO Training Course on Child Growth Assessment and Cole, T. J., 1990. European journal of clinical nutrition, 44(1), pp. 45-60.

### Infant Age

The method of the present invention may comprise using the infant age to determine the personalised DHA supplementation dose. Suitably, the infant age is used to determine the mother's current DHA intake and/or the mother's target DHA intake, which are subsequently used to determine the personalised DHA supplementation dose

The infant age may be given in any measurement unit, suitably days.

### Maternal BMI

The method of the present invention may comprise using the maternal BMI to determine the personalised DHA supplementation dose. Suitably, the maternal BMI is used to determine the mother's current DHA intake and/or the mother's target DHA intake, which are subsequently used to determine the personalised DHA supplementation dose

The method of the present invention may comprise determining the maternal BMI prior to determining the personalised DHA supplementation dose. The method of the present invention may comprise determining the maternal BMI and using this to determine the personalised DHA supplementation dose.

Body mass index (BMI) is a value derived from the mass and height of a person. The BMI is defined as the body mass divided by the square of the body height, and is universally expressed in units of kg/m². The maternal BMI may be determined by any method known in the art for example using a BMI table or calculator.

The maternal BMI may be given in any measurement unit, suitably kg/m².

### Other Parameters

The method of the present invention is not limited to using only the parameters recited herein. Additional parameters may enhance the performance of the method. The method of the present invention may comprise one or more additional parameters (e.g. mother age, infant weight and the like) to determine the personalised DHA supplementation dose. Suitably, the one or more additional parameters are used to determine the mother's current DHA intake and/or the mother's target DHA intake, which are subsequently used to determine the personalised DHA supplementation dose.

Any suitable additional parameter may be used, for example any parameter that has a strong relationship to the DHA intake of the mother. Such parameters may be determined by the skilled person.

The method of the present invention is not limited to a specific population, however some heterogeneity between populations is observed. Limiting the method by population may enhance the performance of the method.

The method of the present invention may be used to determine the personalised DHA supplementation dose for a specific population. For example, the population may be defined by country/region, genetic background, diet and/or lifestyle.

In some embodiments, the population is defined by country/region. For example, the population may be from UK, USA, Canada, Australia, Hong Kong, China, or Mexico.

### Mother's DHA Intake

The personalised DHA supplementation dose may be determined using the mother's current DHA intake and the mother's target DHA intake.

As used herein, the “mother's current DHA intake” is the mother's current intake of DHA from all dietary sources, e.g. from oily fish, algae, seeds etc.

The mother's current DHA intake may be determined at any time during which the mother is breastfeeding the infant. Preferably, the mother's current DHA intake is determined at the same time as the mother's milk DHA level. Suitably, the mother's current DHA intake may be determined at any time between 0-2 years post-partum, or about 0-120 days post-partum. In some embodiments, the mother's current DHA intake is the mother's DHA intake at about 0-3 days post-partum (e.g. about 0 days post-partum, about 1 day post-partum, about 2 days post-partum, or about 3 days post-partum). Suitably, the mother's current DHA intake is the mother's DHA intake at about 2 days post-partum.

As used herein, the “mother's target DHA intake” is the mother's intake of DHA from all dietary sources that is required to meet mother's target milk DHA level.

The mother's target DHA intake may be determined at any time during which the mother is breastfeeding the infant. Suitably, the mother's target DHA intake may be determined at any time between 0-2 years post-partum, or about 0-120 days post-partum. In some embodiments, the mother's target DHA intake is determined at about 0-3 days post-partum (e.g. about 0 days post-partum, about 1 day post-partum, about 2 days post-partum, or about 3 days post-partum). Suitably, the mother's target DHA intake is determined at about 2 days post-partum.

The mother's DHA intake may be for any time interval. For example, the mother's DHA intake may be a daily intake, a monthly intake, or a yearly intake. In some embodiment, the mother's DHA intake is a daily intake.

The mother's current DHA intake and/or the mother's target DHA intake may be given in any measurement unit. For example, the mother's DHA intake may be given in mg. In some embodiments, the mother's DHA intake is given in mg/day.

The mother's current DHA intake and/or the mother's target DHA intake may be determined using the one or more parameters described herein. Preferably, both the mother's current DHA intake and the mother's target DHA intake are determined using the one or more parameters described herein.

The mother's current DHA intake and/or the mother's target DHA intake may be determined using a regression equation. For example, the method of the present invention may comprise determining the mother's current DHA intake and/or the mother's target DHA intake using a regression equation relating the mother's DHA intake to the one or more parameters described herein.

The one or more parameters may be transformed prior to using as a variable in the regression equation. Any suitable transformation may be used. For example, a log transformation may be used to address the skewed distribution of a parameter. Any measurement unit may be used.

As used herein, a “regression equation” relates a dependent variable (e.g. mother's DHA intake) to one or more independent variables (e.g. mother's milk DHA level, maternal BMI, infant weight-for-age z-score, infant age, and the like).

In some embodiments, the regression equation is a multiple regression equation.

As used herein, a “multiple regression equation” relates a dependent variable (e.g. mother's DHA intake) to two or more independent variables (e.g. mother's milk DHA level, maternal BMI, infant weight-for-age z-score, infant age, and the like).

In some embodiments, the regression equation is a linear regression equation.

A “linear regression equation” has an equation of the form Y=a+bX, where X is the independent variable and Y is the dependent variable. The slope of the line is b, and a is the intercept.

In some embodiments, the regression equation is a multiple linear regression equation.

A “multiple linear regression equation” has an equation of the form Y=a+b₁X₁+b₂X₂+b₃X₃+ . . . +bₙXₙ, where Xₙ is an independent variable and Y is the dependent variable. The regression coefficient for each independent variable is bₙ, and a is the intercept.

In some embodiments, the regression equation relates the mother's current DHA intake and/or the mother's target DHA intake to the mother's milk DHA level and one or more infantile parameters, and optionally the maternal BMI.

In some embodiments, the regression equation relates the mother's current DHA intake and/or the mother's target DHA intake to the mother's milk DHA level and infant weight-for-age z-score and/or infant age, and optionally the maternal BMI.

In some embodiments, the regression equation relates the mother's current DHA intake and/or the mother's target DHA intake to the mother's milk DHA level, maternal BMI, infant weight-for-age z-score and infant age.

In some embodiments, the regression equation has an equation of the form:

log(mother's current DHA intake or mother's target DHA intake)=a+b1*log(mother's milk DHA level or mother's target milk DHA level)+b2*log(maternal BMI)+b3*infant weight-for-age z-score+b4*infant age

wherein the intercept, a, and regression coefficients, b1, b2, b3, b4 may each independently take any value, including zero. In some embodiments, a, b1, b2, b3, b4 each independently take any value, excluding zero.

For example, the mother's current DHA intake may be determined using:

log(mother's current DHA intake)=a+b1*log(mother's milk DHA level)+b2*log(maternal BMI)+b3*infant weight-for-age z-score+b4*infant age

For example, the mother's target DHA intake may be determined using:

log(mother's target DHA intake)=a+b1*log(mother's target milk DHA level)+b2*log(maternal BMI)+b3*infant weight-for-age z-score+b4*infant age

Each of the intercept and coefficients can be readily determined using any statistical method known in the art. For example, the intercept and coefficients may be determined by multiple linear regression. Such routine statistical methods may include multiple linear regression with bootstrapping. It is possible to obtain the same estimates with generalised linear or additive models or any other regression-related model with various estimation algorithms, for example, elastic net, lasso, Bayesian approach etc.

The statistical method may be performed on a training dataset. As used herein, a training dataset is a dataset of examples used to fit the parameters or variables.

Any suitable training dataset may be used to perform the statistical method. For example, the training dataset may be obtained from mother's in which the mother's dietary intake of DHA, and the one or more parameters described herein (e.g. mother's milk DHA level, infant weight-for-age z-score, infant age, maternal BMI) are measured.

The values of the intercept and coefficients typically depends on the training dataset used. For example, depending on the country/region, genetic background, diet and/or lifestyle of the training dataset population. Each of the intercept and coefficients can be readily determined for a particular subject cohort.

### Personalised DHA Supplementation Dose

The mother's current DHA intake and/or the mother's target DHA intake may be used to determine the mother's personalised DHA supplementation dose.

The personalised DHA supplementation dose may be for any time interval. For example, the personalised DHA supplementation dose may be a daily dose, a monthly dose, or a yearly dose. In some embodiment, the DHA supplement dose is a daily dose.

The personalised DHA supplementation dose may be given in any measurement unit. For example, the personalised DHA supplementation dose may be given in mg/day.

The personalised DHA supplementation dose may be determined using the difference between the mother's target DHA intake and the mother's current DHA intake.

Suitably, the difference is converted to the personalised DHA supplementation dose using a conversion factor. The bioavailability of DHA from a DHA supplement may be lower than from dietary intake (see e.g. Maki, K. C. and Dicklin, M. R., 2019. Current Opinion in Clinical Nutrition & Metabolic Care, 22(2), pp. 116-123). Therefore, the difference between target and current DHA intake may be increased by a suitable conversion factor to take account of the lower bioavailability of the DHA and to provide a suitable personalised DHA supplementation dose.

The conversion factor may be any suitable conversion factor known in the art and may be specific for the population. For example, Smith S, et al. Prostaglandins Leukot Essent Fatty Acids. 2018 October; 137: 1-4 found that in US mother's 250 mg/day DHA from a fish oil DHA supplement corresponds to about 100 mg/day DHA from diet, in terms of association with the mother's milk DHA levels, which corresponds to a conversion factor of about 2.5.

In some embodiments, the personalised DHA supplementation dose is determined using the following equation:

mother's personalised DHA supplementation dose=(mother's target DHA intake−mother's current DHA intake)*conversion factor.

In some embodiments, the conversion factor is from about 2 to about 3. In some embodiments, the conversion factor is about 2.5. For example, in some embodiments, the personalised DHA supplementation dose is determined using the following equation:

mother's personalised DHA supplementation dose=(mother's target DHA intake−mother's current DHA intake)*2.5.

For example, if the mother's current DHA intake is determined to be 42 mg/day and the mother's target DHA intake is determined to be 100 mg/day, then the mother's personalised DHA supplementation dose may be determined to be 145 mg/day using the above equation.

The personalised DHA supplementation dose may be rounded up or down. For example, the personalised DHA supplementation dose may be rounded to the nearest DHA supplement unit dose. For example, the personalised DHA supplementation doses may be rounded to the nearest 5 mg, 10 mg, 20 mg, 25 mg, 50 mg, 100 mg, or 200 mg.

Preferably, the personalised DHA supplementation dose may be rounded up. For example, the personalised DHA supplementation dose may be rounded up to the nearest DHA supplement unit dose. In some embodiments, the personalised DHA supplementation doses is rounded up to the nearest 5 mg, 10 mg, 20 mg, 25 mg, 50 mg, 100 mg, or 200 mg.

In some embodiments, the personalised DHA supplementation dose is rounded up to the nearest 200 mg. For example, if the DHA supplementation dose is 145 mg/day, the DHA supplementation dose can be rounded up to 200 mg/day.

A DHA+EPA dose of up to 3,000 mg per day is generally considered safe. The personalised DHA supplementation dose may be between 0 mg/day and 3,000 mg/day.

If the mother's milk DHA level already meets the DHA requirements of the mother's infant then no supplementation may be required, other than any normal DHA maintenance dose (e.g. a minimum daily intake of 200 mg/day DHA, as recommended in Koletzko, B., et al., 2008. Journal of perinatal medicine, 36(1), pp. 5-14.)

In some embodiments, if the mother's milk DHA level already meets the DHA requirements of the mother's infant then the personalised DHA supplementation dose is a DHA maintenance dose. For example, if the mother's milk DHA level already meets the DHA requirements of the mother's infant then the DHA maintenance dose may be from 0 mg/day to 400 mg/day. Preferably, if the mother's milk DHA level already meets the DHA requirements of the mother's infant then the DHA maintenance dose is 200 mg/day.

In some embodiments, if the mother's milk DHA level meets a DHA target threshold (e.g. at least about 0.3 g DHA per 100 g fatty acids, at least 0.37 g DHA per 100 g fatty acids, or at least about 0.7 g DHA per 100 g fatty acids) then the personalised DHA supplementation dose is a DHA maintenance dose (e.g. from 0 mg/day to 400 mg/day or 200 mg/day). For example, if the mother's milk DHA level is equal to or greater than 0.37 g DHA per 100 g fatty acids then the personalised DHA supplementation dose may be a DHA maintenance dose (e.g. from 0 mg/day to 400 mg/day or 200 mg/day). Preferably, if the mother's milk DHA level is at least about 0.37 g DHA per 100 g fatty acids then the DHA maintenance dose is 200 mg/day.

In some embodiments, if the mother's milk DHA level does not meet a DHA target threshold (e.g. at least about 0.3 g DHA per 100 g fatty acids, at least 0.37 g DHA per 100 g fatty acids, or at least about 0.7 g DHA per 100 g fatty acids) then the personalised DHA supplementation dose is determined using the method described herein. Preferably, if the mother's milk DHA level less than about 0.37 g DHA per 100 g fatty acids then the personalised DHA supplementation dose is determined using the method described herein.

Suitably, the mother's target milk DHA level exceeds the DHA target threshold. This may maximise the chance for the mother's milk DHA level to reach and/or exceed the DHA requirements of the mother's infant. For example, if the DHA target threshold is at least about 0.37 g DHA per 100 g fatty acids then the personalised DHA supplementation dose may be determined using a mother's target milk DHA level of 0.7 g DHA per 100 g fatty acid.

### Method for Providing a Personalised DHA Supplementation Dose

In one aspect, the present invention provides a method for providing a personalised DHA supplementation dose to be taken by a mother to meet the DHA requirements of the mother's infant.

The personalised DHA supplementation dose may be determined by any method described herein. The personalised DHA supplementation dose may be administered in the form of a DHA supplement.

Docosahexaenoic acid (DHA) (also known as Doconexent, cervonic acid and (4Z,7Z,10Z,13Z,16Z,19Z)-4,7,10,13,16,19-Docosahexaenoic acid) is an omega-3 fatty acid with the following structure:

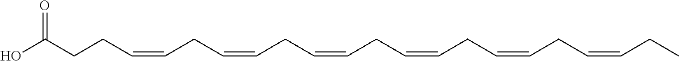 [SMILES: CC/C=C\C/C=C\C/C=C\C/C=C\C/C=C\C/C=C\CCC(=O)O]

The DHA may be derived from any source, for example fish oil and/or algal oil. For example, the DHA supplement may be a fish oil supplement or an algal oil supplement as described in Ryan, L. and Symington, A. M., 2015. Journal of functional foods, 19, pp. 852-858.

A “supplement” or “dietary supplement” may be used to complement the nutrition of an individual (it is typically used as such but it might also be added to any kind of compositions intended to be ingested).

The DHA supplement may be provided in the form of unit doses. For example, each unit dose may comprise about 50 mg, about 100 mg, or about 200 mg DHA. In some embodiments, each unit dose comprises about 200 mg DHA.

The DHA supplement may be in the form of, for example, tablets, capsules, pastilles or a liquid. Suitably, the DHA supplement is in the form of a capsule, for example a gelatin capsule.

The DHA supplement may comprise further components in addition to DHA. For example, the DHA supplement may comprise other poly-unsaturated fatty acids (PUFAs) such as Eicosapentaenoic acid (EPA) and Docosapentaenoic acid (DPA) and the like. For example, a fish oil supplement may comprise about 22% DHA and 30% EPA by weight total fatty acids (as described in Barrow, C. J., et al., 2009. Journal of Functional Foods, 1(1), pp. 38-43). For example, an algal oil supplement may comprise about 40% DHA, 15% DPA and 2.5% EPA by weight total fatty acids (as described in Arterburn, L. M., et al., 2007. Lipids, 42(11), p. 1011).

Suitably, the DHA supplement does not comprise appreciable amounts of any other PUFA. For example, an algal oil supplement may comprise about 40% DHA and no appreciable amounts of any other PUFA by weight total fatty acids (as described in Arterburn, L. M., et al., 2007. Lipids, 42(11), p. 1011).

The DHA supplement may contain an organic or inorganic carrier material suitable for oral or parenteral administration as well as vitamins, minerals trace elements and other micronutrients.

### Method of Improving the Health and/or Development of an Infant

In one aspect, the present invention provides a method of promoting development and/or improving the health of an infant, the method comprising administering a personalised DHA supplementation dose to the infant's mother.

In one aspect, the present invention provides a personalised DHA supplementation dose, for use in a method of promoting development and/or improving the health of an infant, the method comprising administering a personalised DHA supplementation dose to the infant's mother.

The personalised DHA supplementation dose may be determined by any method described herein. The personalised DHA supplementation dose may be administered in the form of a DHA supplement.

As used herein, “promoting development” of an infant includes promoting the development of any of the mental or physical characteristics of the infant including cognitive development, visual acuity, motor skills, language and communication skills, immune system development, and physical growth.

As used herein, “improving the health” of an infant includes improving the survival and long-term well-being of the infant, for example reducing the risk of the infant developing a chronic disease.

DHA plays important roles in health and development. DHA is especially important for the brain and retina, where it rapidly accumulates during the early years of life and plays an important role in the development of visual and cognitive function. DHA is also an important precursor and messenger for a variety of biological processes, particularly in relation to cerebral, cardiovascular and immune functions. DHA also plays a role in the prevention or treatment of common chronic diseases that may lead to significant morbidity and mortality. (Agostoni, C., et al., 2016. Sight and Life, 30, pp. 82-6).

In particular, a high milk DHA supply to very low birth weight infants may enhance early visual and cognitive development and reduce adverse events include severe developmental delay, bronchopulmonary dysplasia, necrotizing enterocolitis and allergies (Koletzko, B., 2017. Clinics in perinatology, 44(1), pp. 85-93).

### Cognitive Development, Visual Acuity and Motor Skills

DHA plays important roles throughout the body and is essential for maintaining the structure and function of the brain and eye. Fetal development and infancy are key windows during which sufficient DHA levels are necessary for optimal mental and visual development and performance in later life (Calder, P. C., 2016. Docosahexaenoic acid. Annals of Nutrition and Metabolism, 69(Suppl. 1), pp. 8-21). In particular, DHA supplementation of mothers during the complementary feeding period may be effective in improving neurofunctional and visual performance (Agostoni, C., 2010. Early human development, 86(1), pp. 3-6).

In one aspect, the present invention provides a method of promoting cognitive development, visual acuity, and/or motor skill development comprising administering the personalised DHA supplementation dose to the infant's mother.

In one aspect, the present invention provides a personalised DHA supplementation dose, for use in a method of promoting cognitive development, visual acuity, and/or motor skill development comprising administering the personalised DHA supplementation dose to the infant's mother.

### Immune System Development

The early availability of LC-PUFA such as DHA has been associated with immune functions and the likelihood of the development of allergies and infections (Koletzko, B., et al., 2014. Annals of Nutrition and Metabolism, 65(1), pp. 49-80).

In one aspect, the present invention provides a method of promoting immune system development, comprising administering the personalised DHA supplementation dose to the infant's mother.

In one aspect, the present invention provides a personalised DHA supplementation dose, for use in a method of promoting immune system development comprising administering the personalised DHA supplementation dose to the infant's mother.

In one aspect, the present invention provides a method of reducing an infant's risk of developing allergies, comprising administering the personalised DHA supplementation dose to the infant's mother.

In one aspect, the present invention provides a personalised DHA supplementation dose, for use in a method of reducing an infant's risk of developing allergies, comprising administering the personalised DHA supplementation dose to the infant's mother.

### Risk of Becoming Overweight or Obese

Rapid weight gain in infancy and low levels of n-3 long chain polyunsaturated fatty acids (LC-PUFA) such as DHA at birth are associated with increased adiposity later in life (O'Tierney-Ginn, P. F., et al., 2017. Journal of developmental origins of health and disease, 8(4), p. 474).

In one aspect, the present invention provides a method of reducing an infant's risk of becoming overweight or obese in infancy and/or early childhood, comprising administering the personalised DHA supplementation dose to the infant's mother.

In one aspect, the present invention provides a personalised DHA supplementation dose, for use in a method of reducing an infant's risk of becoming overweight or obese in infancy and/or early childhood, comprising administering the personalised DHA supplementation dose to the infant's mother.

### Computer Program and Computer-Readable Medium

The method described herein for determining a personalised DHA supplementation dose to be taken by a mother may be a computer-implemented method.

In one aspect, the present invention provides a computer-implemented method for determining a personalised DHA supplementation dose to be taken by a mother to meet the DHA requirements of the mother's infant comprising:
(i) using one or more parameters, preferably the mother's milk DHA level and one or more infantile parameters, to determine the mother's current DHA intake and/or the mother's target DHA intake; and
(ii) using the mother's current DHA intake and the mother's target DHA intake to determine the personalised DHA supplementation dose.

In one aspect, the present invention provides a data processing system comprising means for determining a personalised DHA supplementation dose to be taken by a mother to meet the DHA requirements of the mother's infant, given one or more parameters.

In one aspect, the present invention provides a data processing apparatus comprising a processor configured to determine a personalised DHA supplementation dose to be taken by a mother to meet the DHA requirements of the mother's infant, given one or more parameters.

In one aspect, the present invention provides a computer program comprising instructions which, when the program is executed by a computer, cause the computer to determine a personalised DHA supplementation dose to be taken by a mother to meet the DHA requirements of the mother's infant, given one or more parameters.

In one aspect, the present invention provides a computer-readable medium comprising instructions which, when executed by a computer, cause the computer to determine a personalised DHA supplementation dose to be taken by a mother to meet the DHA requirements of the mother's infant, given one or more parameters.

The parameters may be any parameters described herein. The personalised DHA supplementation dose may be determined by any method described herein.

In one aspect, the present invention provides a data processing system comprising means for carrying out the method of the invention.

In one aspect, the present invention provides a data processing apparatus comprising a processor configured to perform the method of the invention.

In one aspect, the present invention provides a computer program comprising instructions which, when the program is executed by a computer, cause the computer to carry out the method of the invention.

In one aspect, the present invention provides a computer-readable medium comprising instructions which, when executed by a computer, cause the computer to carry out the method of the invention.

In one aspect, the present invention provides a computer-readable data carrier having stored thereon the computer program of the invention.

In one aspect, the present invention provides a data carrier signal carrying the computer program of the invention.

### EXAMPLES

The invention will now be further described by way of examples, which are meant to serve to assist the skilled person in carrying out the invention and are not intended in any way to limit the scope of the invention.

### Example 1—Algorithm to Predict the Amount of DHA Intake Requisite to Achieve and Maintain Target Levels of DHA in Human Milk

### Developing the Algorithm

a. Data Cleaning and Preparation

Log-transformation is used to address the skewed distribution of dietary DHA intake amount and milk DHA levels.

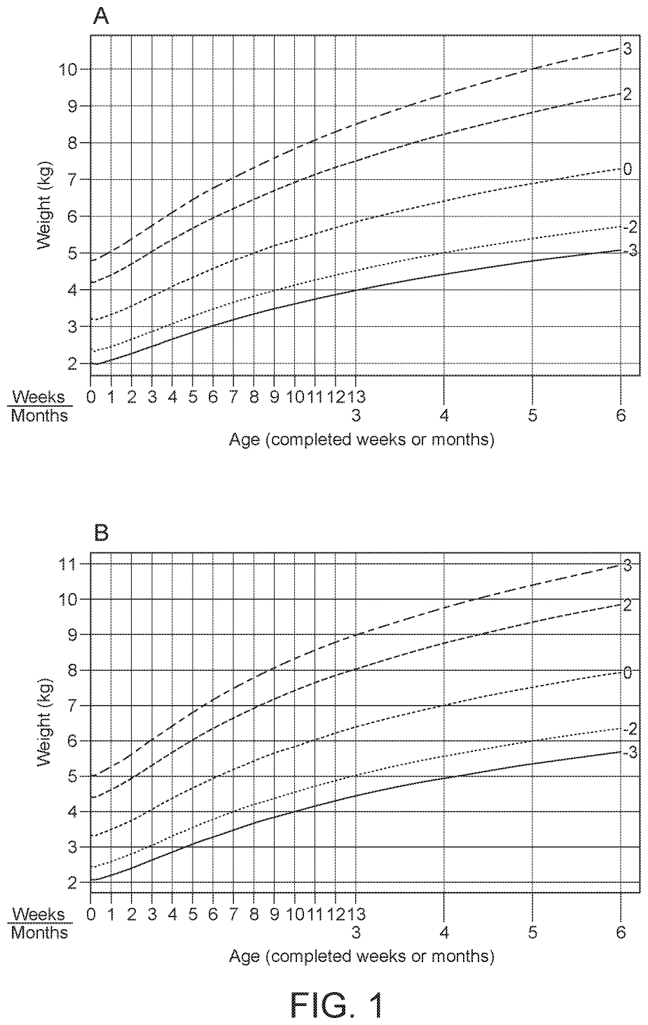

Baby's weight for age z-score is calculated from baby's weight, sex and using the standard deviation from WHO (FIG. 1).

b. Feature Selection

Filter methods including univariate analysis and decision tree are adopted to remove non-informative or redundant variables and to select those predictors with strong relationship to the response variable. The selected variables are as follows:
1) Human milk DHA: DHA levels of milk sample collected at registration, express as percent of total fatty acids
2) Maternal BMI: Mother's BMI when milk sample collected, expressed as kg/m2
3) Baby's weight for age z-score: Z-score expresses the anthropometric value as a number of standard deviations below or above the reference value.
4) Baby's age: baby's age in days

Variance inflation factors (VIF) are also checked to avoid severe multicollinearity, the VIF scores are shown below in Table 1 and are close to 1, indicating that there is no correlation among the referred predictor and the remaining predictor variables, hence the variance of this predictor is not inflated and the multicollinearity could be ignored.

TABLE 1
VIF scores of model built

| Features | VIF scores |
|---|---|
| Log transformed | 1.078 |
| milk DHA | |
| Baby's weight for | 1.019 |
| age z-score | |
| Baby's age | 1.094 |

c. Model Construction and Evaluation

With the variables (milk DHA, maternal BMI, baby's weight for age z-score and baby's age) identified from last step serving as model features, an ordinary linear regression model is built to describe the dependency between dietary intake amount of DHA and independent variables.

The final algorithm will depend on the data used (e.g. depending on the country/region, genetic background, diet and/or lifestyle of the training dataset population). For example, the final algorithm may be as follows:

log(DHA_intake/maternal_bmi)˜b1*log(milk_DHA)+b2*baby_weight_age_z-score+b3*baby age−a

wherein the intercept, a, and regression coefficients, b1, b2, b3, may each independently take any value, including zero

The algorithm can be used to determine the mother's current DHA intake (based on the mother's milk DHA level) and the mother's target DHA intake (based on the mother's target milk DHA level).

The literature describes a range of different target milk DHA levels. Any target milk DHA level could be used, for example:
a. 0.3% is shown to have improved health outcomes of infants (Harris and Black, 2015), also minimum recommended by American Academy of Pediatrics.
b. 0.37% is the global average of human milk DHA (Fu et al., 2016).
c. 1.0% is based on ecological evidence in natural setting to prevent excessive depletion in infants and mothers for optimal outcomes (Luxwolda et al., 2014).
d. 0.7% represents linear center of 0.37-1% range. Equivalent to 85^{th} percentile of LIFE data from European.

Preferably, 0.7 g DHA per 100 g fatty acids (option d) may be selected as the target milk DHA level to maximise the chance for mother's milk DHA to reach and/or exceed the global average.

The personalised recommendation may then be determined by using the following equation:

mother's personalised DHA supplementation dose=(mother's target DHA intake−mother's current DHA intake)*conversion factor

Any suitable conversion factor could be used. A conversion factor of 2.5 was inferred to link the dose conversion from dietary intake DHA to fish oil DHA. Smith S, et al. Prostaglandins Leukot Essent Fatty Acids. 2018 October; 137: 1-4 found that 250 mg/day DHA from fish oil DHA supplementation corresponds to about 100 mg/day DHA from diet, in terms of association with the mother's milk DHA levels.

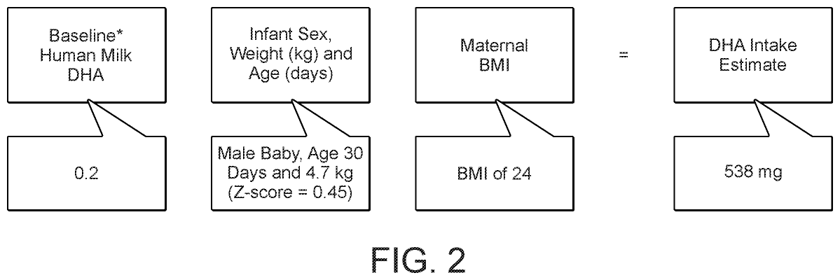

An example of personalised recommendation using the method described above is shown in FIG. 2.

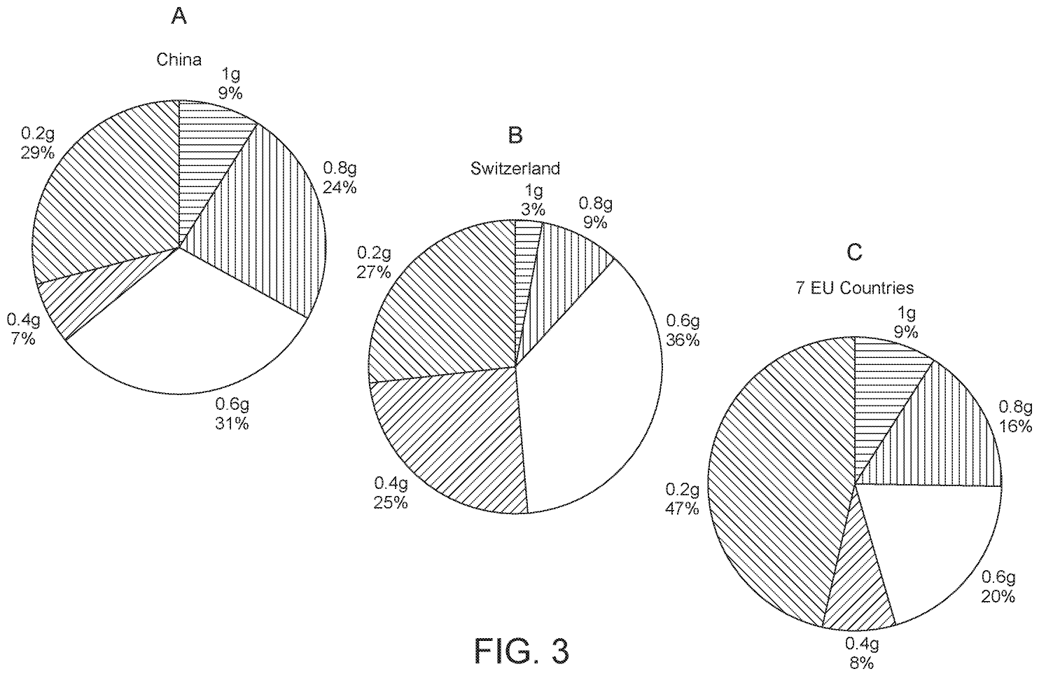

In order to validate the model new data collected from clinical trial done in other country (China, Switzerland) instead of the seven countries mentioned in the first section, is fed into the built model for DHA intake amount prediction. As shown in FIG. 3, the predicted DHA intake amount are considered as reasonable from scientific perspective.

d. Personalised DHA Supplementation Dose.

In order to provide personalised DHA supplementation dose, the following information needs to be obtained from the mothers (in addition to determining the maternal milk DHA level):
1) Mother birth date (Day/Month/Year)
2) Mother weight (in kg or lbs)
3) Mother height (in cm or feet and inches)
4) Baby birth date (Day/Month/Year)
5) Baby sex (m/f)
6) Baby weight (in kg or lbs)

Among these entries, maternal milk DHA level is compulsory and others may be optional. If any of the optional information is missing, common values can be used to facilitate the dose estimate. For example, a baby's weight for age z-score of 0 could be used, a maternal BMI of 21.5 kg/m² could be used, and a baby age of 2 months could be used if any of these entries were missing. These optional variables will provide a more personalised recommendation.

If the maternal milk DHA level is above a threshold value then a DHA maintenance dose may be recommended. For example, if the maternal milk DHA level is greater than or equal to 0.37% then a DHA maintenance dose of 200 mg/day may be recommended. Otherwise, if the maternal milk DHA level is less than 0.37% then the personalised DHA supplementation dose may be determined using the method described above, using a target milk DHA level of e.g. 0.7% to maximise the chance for mother's milk DHA to reach and/or exceed 0.37%.

The ceiling rounding rule can be applied to recommend the final DHA supplementation dose. If the estimated DHA supplementation dose by the model is higher than the maintenance dose and sits between two supplement product doses, then the higher dose can be recommended. For example, if a calculated recommendation from the algorithm is 420 mg, the recommended dose informed to consumers can be 600 mg since the available supplement product doses are 200 mg, 400 mg, 600 mg, 800 mg and 1000 mg. If more supplement product doses are available, further precision on personalisation can be achieved.

### Data for Developing the Algorithm

### Existing Human Milk Data

Existing human milk data from a study was used to develop the algorithm, i.e. as training data. Any other data set could have been used to develop the algorithm.

The study (Atlas, Europe) was conducted in seven countries across Europe (France, Italy, Norway, Portugal, Romania, Spain, and Sweden) as a longitudinal, observational, cohort in which human milk as well as multiple maternal and infant parameters were collected at six time points post-partum (2 (0-3) d, 17±3 d, 30±3 d, 60±5 d, 90±5 d, and 120±5 d). The study was registered at www.clinicaltrials.gov with the identifier NCT01894893. Pregnant women were recruited before delivery, generally during the last trimester of pregnancy.

Inclusion criteria for this study were: (i) pregnant women between ages of 18 and 40 years; (ii) pre-pregnancy BMI between 19 and 29, inclusive; (iii) intention to breastfeed at least until 4 month post-partum and (iv) agreed to the study protocol and signed the informed consent form.

Exclusion criteria for this study were: (i) currently participating in another trial; (ii) presenting conditions that contraindicate breastfeeding; (iii) having medical conditions or on medications for conditions such as metabolic and cardiovascular abnormalities; (iv) dietary problems such as anorexia or bulimia and (v) not being able to comply to the study procedures.

Maternal data included: demography, anthropometry, medical history, history of dietary supplements, three-day food diaries.

Infant data included: demography, anthropometry, history of medication use, body composition (one center in France and one in Sweden), infant HM intake diary (three centers in France only).

A data set with additional parameters may enhance the performances of the algorithm. For example, genetic data, dietary data, lifestyle data, etc.

Heterogeneity between countries and population behaviors is observed. Using a data set for each population may enhance the model performance and enhance the performance of the algorithm for that population.

### Published Evidence

16 external publications were analysed for development of the algorithm, which included omega-3 dietary intake and supplement intake correlation with human milk omega-3. Any other publications could have been used to develop the algorithm. Publications included:
Smith S, et al. Prostaglandins Leukot Essent Fatty Acids. 2018 October; 137: 1-4—Reference for conversion rate between food and supplement DHA
Stoutjesdijk E, et al. Prostaglandins Leukot Essent Fatty Acids. 2018 January; 128: 53-61—Reference for linkage between supplement DHA intake and milk DHA
Liu M J, et al. Nutrients. 2016 May 20; 8(5): 312.—Reference for linkage between diet DHA intake and milk DHA
Wu W C, et al. Nutrients. 2020 February 20; 12(2): 543—Reference for linkage between diet and supplement DHA intake and milk DHA

### Embodiments

Various preferred features and embodiments of the present invention will now be described with reference to the following numbered paragraphs (paras).

1. A method for determining a personalised docosahexaenoic acid (DHA) supplementation dose to be taken by a mother to meet the DHA requirements of the mother's infant, wherein the method comprises using the mother's milk DHA level and one or more infantile parameters to determine the personalised DHA supplementation dose.

2. The method according to para 1, wherein the one or more infantile parameters comprise infant weight-for-age z-score and/or infant age.

3. The method according to para 1 or para 2, wherein the method further comprises using maternal BMI to determine the personalised DHA supplementation dose.

4. The method according to any preceding para, wherein the method comprises using mother's milk DHA level, maternal BMI, infant weight-for-age z-score and infant age to determine the personalised DHA supplementation dose.

5. The method according to any preceding para, wherein the method further comprises using one or more additional parameters to determine the personalised DHA supplementation dose, preferably wherein the one or more additional parameters are selected from one or more of the country or region, genetic data, maternal age, dietary data, and lifestyle data.

6. The method according to any preceding para, wherein the DHA requirements of the mother's infant is a mother's milk DHA level of at least 0.3 g DHA per 100 g fatty acids, at least 0.37 g DHA per 100 g fatty acids, or at least 0.7 g DHA per 100 g fatty acids.

7. The method according to any preceding para, wherein the DHA requirements of the mother's infant is a mother's milk DHA level of 0.3-1.0 g DHA per 100 g fatty acids, 0.37-1.0 g DHA per 100 g fatty acids, 0.7-1.0 g DHA per 100 g fatty acids, or about 0.7 g DHA per 100 g fatty acids, preferably wherein the DHA requirement of the mother's infant is a mother's milk DHA level of about 0.7 g DHA per 100 g fatty acids.

8. The method according to any preceding para, wherein the method comprises:
(i) using the mother's milk DHA level and one or more infantile parameters to determine the mother's current DHA intake and/or the mother's target DHA intake; and
(ii) using the mother's current DHA intake and the mother's target DHA intake to determine the personalised DHA supplementation dose.

9. The method according to para 8, wherein the mother's current DHA intake and/or the mother's target DHA intake are determined using a regression equation, preferably wherein the regression equation is a multiple linear regression equation.

10. The method according to para 9, wherein the regression equation is:

log(mother's current DHA intake or mother's target DHA intake)=a+b1*log(mother's milk DHA level or mother's target milk DHA level)+b2*log(maternal BMI)+b3*infant weight-for-age z-score+b4*infant age,

wherein a, b1, b2, b3, b4 are determined by multiple linear regression.

11. The method according to any one of paras 8-10, wherein the personalised DHA supplementation dose is determined using the difference between the mother's target DHA intake and the mother's current DHA intake.

12. The method according to any one of paras 8-11, wherein the personalised DHA supplementation dose is determined using the following equation:

personalised DHA supplementation dose=(mother's target DHA intake−mother's current DHA intake)*conversion rate.

13. The method according to para 12, wherein the conversion rate is from about 2 to about 3, or about 2.5.

14. The method according to any preceding para, wherein the personalised DHA supplementation dose is a daily dose.

15. The method according to any preceding para, wherein the personalised DHA supplementation dose is rounded up to the nearest 5 mg, 10 mg, 20 mg, 25 mg, 50 mg, 100 mg, or 200 mg, preferably wherein the personalised DHA supplementation dose is rounded up to the nearest 200 mg.

16. A method for determining a personalised DHA supplementation dose to be taken by a mother to meet the DHA requirements of the mother's infant, wherein the method comprises:
(i) determining the mother's milk DHA level; and
(ii) determining the personalised DHA supplementation dose using the method according to any one of paras 1-15.

17. A method for providing a personalised DHA supplementation dose to be taken by a mother to meet the DHA requirements of the mother's infant, wherein the method comprises:
(i) determining the personalised DHA supplementation dose using the method according to any one of paras 1-16; and
(ii) administering the personalised DHA supplementation dose to the mother.

18. A method of improving the health and/or development of an infant, the method comprising administering a personalised DHA supplementation dose to the infant's mother, wherein the personalised DHA supplementation dose is determined using the method according to any one of paras 1-16.

19. A personalised DHA supplementation dose, for use in a method of promoting development and/or improving the health of an infant, the method comprising administering a personalised DHA supplementation dose to the infant's mother, wherein the personalised DHA supplementation dose is determined using the method according to any one of paras 1-16.

20. The method according to para 18, or the personalised DHA supplementation dose for use according to para 19, wherein cognitive development, visual development, motor skill development and/or immune system development of the infant is improved.

21. The method according to para 18 or 20, or the personalised DHA supplementation dose for use according to para 19 or 20, wherein the infant's risk of becoming overweight or obese in infancy and/or early childhood is reduced.

22. A computer program comprising instructions which, when the program is executed by a computer, cause the computer to carry out the method according to any one of paras 1-16.

23. A computer program comprising instructions which, when the program is executed by a computer, cause the computer to determine a personalised DHA supplementation dose to be taken by a mother to meet the DHA requirements of the mother's infant, given the mother's milk DHA level and one or more infantile parameters.

24. A computer-readable medium comprising instructions which, when executed by a computer, cause the computer to carry out the method according to any one of paras 1-16.

25. A computer-readable medium comprising instructions which, when executed by a computer, cause the computer to determine a personalised DHA supplementation dose to be taken by a mother to meet the DHA requirements of the mother's infant, given the mother's milk DHA level and one or more infantile parameters

26. Use of a personalised DHA supplement, wherein the personalised DHA supplement dose is determined using the method according to any one of paras 1-16.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining a personalised docosahexaenoic acid (DHA) supplementation dose to be taken by a mother to meet the DHA requirements of the mother's infant, wherein the method comprises using the mother's milk DHA level and one or more infantile parameters to determine the personalised DHA supplementation dose.

2. The method according to claim 1, wherein the one or more infantile parameters comprise infant weight-for-age z-score and/or infant age.

3. The method according to claim 1, wherein the method further comprises using maternal BMI to determine the personalised DHA supplementation dose.

4. The method according to claim 1, wherein the method further comprises using one or more additional parameters to determine the personalised DHA supplementation dose.

5. The method according to claim 1, wherein the method comprises:
(i) using the mother's milk DHA level and one or more infantile parameters to determine the mother's current DHA intake and/or the mother's target DHA intake; and
(ii) using the mother's current DHA intake and the mother's target DHA intake to determine the personalised DHA supplementation dose.

6. The method according to claim 5, wherein the mother's current DHA intake and/or the mother's target DHA intake are determined using a regression equation, preferably wherein the regression equation is a multiple linear regression equation.

7. The method according to claim 6, wherein the regression equation is:
log(mother's current DHA intake or mother's target DHA intake)=a+b1*log(mother's milk DHA level or mother's target milk DHA level)+b2*log(maternal BMI)+b3*infant weight-for-age z-score+b4*infant age,
wherein a, b1, b2, b3, b4 are determined by multiple linear regression.

8. The method according to claim 5, wherein the personalised DHA supplementation dose is determined using the difference between the mother's target DHA intake and the mother's current DHA intake.

9. The method according to claim 1, wherein the personalised DHA supplementation dose is a daily dose; and/or wherein the personalised DHA supplementation dose is rounded up to the nearest 200 mg.

10. A method for determining a personalised DHA supplementation dose to be taken by a mother to meet the DHA requirements of the mother's infant, wherein the method comprises:
(i) determining the mother's milk DHA level; and
(ii) determining the personalised DHA supplementation dose using the mother's milk DHA level and one or more infantile parameters to determine the personalised DHA supplementation dose.

11-15. (canceled)
